(19) United States
(12) Patent Application Publication
(10) Pub. No.: US 2017/0304207 A1
(43) Pub. Date: Oct. 26, 2017

(54) MANUFACTURE OF A PHARMACEUTICAL PRODUCT

(71) Applicants: National University of Singapore, Singapore (SG); Massachusetts Institute of Technology, Cambridge, MA (US)

(72) Inventors: Saif A. KHAN, Singapore (SG); Reno Antony Louis LEON, Singapore (SG); Abu Zayed Md. BADRUDDOZA, Singapore (SG); Wai Yew WAN, Singapore (SG); T. Alan HATTON, Cambridge, MA (US)

(21) Appl. No.: 15/647,599

(22) Filed: Jul. 12, 2017

Related U.S. Application Data
(63) Continuation of application No. 15/030,537, filed on Apr. 19, 2016.
(60) Provisional application No. 61/894,989, filed on Oct. 24, 2013.

(51) Int. Cl. A61K 9/16; A61K 31/381; A61K 31/00; A61K 9/14; A61K 31/55; A61K 9/113

(52) U.S. Cl. CPC A61K 9/1682; A61K 31/55; A61K 31/381; A61K 31/00; A61K 9/1652; A61K 9/1623; A61K 9/146; A61K 9/145; A61K 9/113

(57) ABSTRACT

An emulsion-based method for the manufacture of a crystalized spherical agglomerate and/or a pharmaceutical product is provided; and crystalized spherical agglomerates and/or pharmaceutical products manufactured thereby.

This application is a continuation patent application claiming priority to U.S. patent application Ser. No. 15/030,537 filed on Apr. 19, 2016, which in turn claims priority as the national stage of international patent application no. PCT/SG2014/000486 filed on Oct. 15, 2014, which in turn claims priority from U.S. Provisional Patent Application No. 61/894,989 filed on Oct. 24, 2013, the disclosures of which are incorporated herein by reference in their entirety.

### TECHNICAL FIELD

The invention concerns an emulsion-based method for the manufacture of a crystalized spherical agglomerate and/or a pharmaceutical product; and crystalized spherical agglomerate and/or a pharmaceutical product manufactured thereby.

### BACKGROUND

Pharmaceutical formulation processes, in which active pharmaceutical ingredients (APIs) are blended with additives and excipients, are crucial downstream operations that dictate the final pharmacokinetic attributes of the product. Presently, most APIs are produced in crystalline form and their formulation typically involves energy intensive downstream operations such as comminution, milling, sieving, blending and granulation, before tableting into the final product.

These steps are necessitated by poorly controlled primary crystallization processes which typically yield large crystals of irregular size and shape. The differently shaped crystals have different physical properties, such as plasto-elasticity and compaction behaviour, which affects the afore downstream operations.

Indeed, particle size is extremely important in the pharmaceutical industry. The size, and hence the surface area of an API in particle form, can be related to the physical, chemical and pharmacologic properties of the drug containing same. Clinically, the particle size of an API can affect its release from dosage forms that are administered orally, parenterally, rectally and topically. The successful formulation of pharmaceuticals; both their physical stability and pharmacologic response also depends on the particle size achieved in the final product.

An alternative manufacturing technique—emulsion-based crystallization—has shown great potential by producing monodispersed, spherical agglomerates (SAs) which possess superior micromeritic properties (such as powder packability and flowability), improved chemical stability and bioavailability, and most importantly, the feasibility of direct compression into tablets.

Furthermore, multiple emulsions (such as double, triple, quadruple, etc. emulsions) which have been known since 1925 offer the potential benefits of multiple liquid domains of different natures (hydrophobic and hydrophilic) and thus flexibility in the choice of ingredients to be formulated are compatible with the mentioned technique. However, one of the main problems associated with the use of multiple emulsions to date is their inherent instability. Multiple emulsions are complex systems as the drops of the dispersed phase themselves contain even smaller dispersed droplets.

Some of the possible instabilities of these systems include: multiple oil drops may coalesce with other oil drops, one or more internal aqueous droplets may be expelled from within an oil droplet in a water/oil/water system, the internal droplets may coalesce before being expelled from within the droplet containing them, and water may pass through the oil phase by diffusion resulting in the gradual shrinkage of the internal droplet.

It is therefore considered that that multiple emulsions whilst having many potential uses are complex and inherently unstable systems.

The invention described herein involves the use of emulsions in a one-step formulation followed by crystallization to form SAs. Our invention circumvents several drawbacks in conventional processing, such as wide size distribution in batch crystallization, de-mixing in blending and challenges in the formulation of hydrophobic and hydrophilic APIs and excipients thereby offering the potential for continuous, sustainable pharmaceutical crystallization coupled with advanced formulations.

### SUMMARY

According to a first aspect of the invention there is provided a method for the manufacture of a pharmaceutical product comprising:
i) dispersing a first pharmacologically active ingredient in a first fluid;
ii) dispersing an excipient in a second fluid;
iii) mixing said first and second fluids with a third carrier fluid to form a multiple emulsion; and
iv) collecting the emulsion on a heated surface and allowing the emulsion to crystalize to form spherical agglomerates.

Preferably, the invention is worked using more than one pharmacologically active ingredient and the method comprises:
i) dispersing a first pharmacologically active ingredient in a first fluid;
ii) dispersing a second pharmacologically active ingredient and an excipient in a second fluid
iii) mixing said first and second fluids with a third carrier fluid to form a multiple emulsion; and
iv) collecting the emulsion on a heated surface and allowing the emulsion to crystalize to form spherical agglomerates.

The invention can involve the co-formulation of a hydrophobic and hydrophilic drugs in the presence of an excipient. The methods of the invention circumvent several energy intensive downstream processes in traditional manufacturing, thereby offering the potential of continuous, sustainable pharmaceutical crystallization coupled with advanced drug formulations which can be used to achieve a multitude of drug delivery objectives.

In a preferred embodiment of the invention said pharmacologically active ingredient is either hydrophobic or hydrophilic.

In a preferred embodiment of the invention said hydrophobic pharmacologically active ingredient is dispersed in a compatible fluid such as a non-aqueous first fluid.

In a preferred embodiment of the invention said hydrophilic pharmacologically active ingredient is dispersed in a compatible fluid such as an aqueous first fluid.

In a further preferred embodiment of the invention said excipient is dispersed in a compatible fluid such as an aqueous or non-aqueous second fluid.

In yet a further preferred embodiment of the invention said hydrophobic pharmacologically active ingredient and excipient is dispersed in a compatible fluid such as a non-aqueous second fluid.

In yet another preferred embodiment of the invention said hydrophilic pharmacologically active ingredient and excipient is dispersed in a compatible fluid such as an aqueous second fluid.

Those skilled in the art will appreciate that the emulsion of the invention may therefore be either a water/oil/water emulsion or an oil/water/oil emulsion.

In yet further preferred embodiments of the invention the method may involve the use of a fourth, fifth, etc. fluid in each of which there is dispersed a further hydrophobic/hydrophilic pharmacologically active ingredient and, optionally, an excipient. In this embodiment of the invention a complex multiple emulsion is formed prior to crystallization on a heated surface.

In yet a further preferred embodiment of the invention said mixing is undertaken by passing the said fluids through a mixing device such as a channel, ideally, a micro channel and so ideally involves the use of a microfluidic device. More preferably still, each fluid is introduced into the microfluidic device via a different channel each one of which converges at a mixing point where the said channels are brought together.

More preferably, the rate of flow of said fluids through said channels is controlled or regulated in accordance with the desired formulation of the pharmacologically active ingredient(s). Thus, in addition to controlling the amount of pharmacologically active ingredient in its compatible fluid one can also control the flow of each fluid through said mixing device thus helping to refine the nature of the formulation.

We have discovered the microfluidic method enables formulations that are nearly impossible to achieve using conventional crystallization methods.

Using the method of the invention we have produced monodispersed microparticles containing crystals of a hydrophobic API embedded within an excipient (‘DE’ i.e. Drug/Excipient formulation), which in turn may also contain a hydrophilic API (‘D²E’ Drug/Drug/Excipient formulation). Whilst the invention has been worked as described, it can be worked using various combinations of hydrophobic/hydrophilic/excipient. For example, monodispersed microparticles containing crystals of a hydrophilic API embedded within an excipient (‘DE’ formulation), which in turn may also contain a hydrophobic API (‘D²E’ formulation) can be produced.

More specifically, using the method of the invention we have produced monodispersed microparticles containing crystals of a hydrophobic model API (5-methyl-2-[(2-nitrophenyl)amino]-3-thiophenecarbonitrile, termed ROY) embedded within a hydrophilic excipient (sucrose) matrix (‘DE’ formulation), which in turn may also contain a hydrophilic model API (glycine) (‘D²E’ formulation).

Further, using the method of the invention we have produced monodispersed microparticles or SAs in the order of 100-300 μm and typically 200 μm.

Furthermore, our method also involves controlling the polymorphic selection of the crystalized spherical agglomerate (SA) via the kinetics of two simultaneous processes occurring within the evaporating emulsion drops containing API-excipient mixtures—(i) liquid-liquid phase separation, which compartmentalizes the API while also providing sites for heterogeneous polymorphic nucleation and (ii) increasing supersaturation of both the API and excipient rich phases, eventually leading to solidification of the excipient, which further facilitates nucleation and crystallization of the API.

We have demonstrated our method using two model hydrophobic APIs—5-methyl-2-[(2-nitrophenyl)amino]-3-thiophenecarbonitrile (also known as ‘ROY’, due to the characteristic color of its polymorphs) and carbamazepine (CBZ), formulated with ethyl cellulose (EC) as excipient. ROY and CBZ both exhibit conformational polymorphism, with ten and four known polymorphs respectively. In our method, an API-excipient solution in the solvent, dichloromethane (DCM) is used to form oil-in-water (O/W) emulsions with an aqueous solution of polyvinyl alcohol (PVA) serving as the continuous phase, in a micro-capillary emulsion generator. This is followed by thin-film evaporation. Remarkably, we are able to control the polymorphic selection by varying solvent evaporation rate, which is simply tuned by the film thickness; fast (film thickness ˜0.5 mm, the thinnest we can achieve without breaking the emulsions) and slow (film thickness ˜2 mm) lead to completely specific and different polymorphic outcomes for both model APIs—yellow (YT04) and orange (OP) for ROY, and form II and form III for CBZ respectively. Our method thus paves the way for simultaneous, bottom-up crystallization and formulation processes coupled with unprecedented polymorphic selection through process driven kinetics. We envision it to form the basis of simple, robust and sustainable process platforms for continuous pharmaceutical drug particle manufacturing.

Accordingly, in a further embodiment the method in part iv) involves collecting the emulsion on a heated surface at a selected film thickness and allowing the emulsion to crystalize to form spherical agglomerates.

Most preferably said film thickness is selected having regard to the desired emulsion droplet size to be produced and so is subject to routine determination by those skilled in the art, however, as exemplified herein a film size between 0.5-2 mm is used to work the invention.

According to a second aspect of the invention there is provided a crystalized spherical agglomerate (SA) manufactured according to the method of the invention comprising at least one pharmacologically active ingredient (API) and an excipient or carrier.

According to a third aspect of the invention there is provided a crystalized spherical agglomerate (SA) comprising at least one pharmacologically active ingredient (API) and an excipient or carrier.

Additionally, or alternatively, said crystalized spherical agglomerate (SA) comprises at least one further pharmacologically active ingredient (API).

In a preferred embodiment of the invention said spherical agglomerate (SA) comprises particles in the order of 100-300 μm and typically 200 μm.

According to a further aspect of the invention there is provided a pharmaceutical comprising the said crystalized spherical agglomerate (SA).

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

Throughout the description and claims of this specification agarose is denoted as AG and heparin as HEP.

### BRIEF DESCRIPTION OF THE DRAWINGS

An embodiment of the present invention will now be described by way of example only with particular reference to the following wherein:

FIG. 1 shows a schematic descriptive of the technique for co-formulation and crystallization of hydrophobic and hydrophilic API using double emulsions. Drug 1 (Red/dark grey) represents a hydrophobic API while Drug 2 (Yellow/light grey) represents a hydrophilic API, dispersed in a matrix comprising of an excipient (Pink/mid grey).

FIG. 2 shows the apparatus and equipment required for working the invention. The components are marked with numbers: 1) double emulsion generation apparatus; 2) heated surface; 3) stereo microscope; 4(a)-(c) syringe pumps; 5) light source.

FIG. 3 shows a schematic of emulsion generation apparatus depicting generation of O₁/W/O₂ (Red/Blue/Yellow) double emulsion drops with multiple (‘n’-in-1) inner O₁ droplets (Red Spheres) using capillary microfluidics, followed by evaporative crystallization of the droplets to form SAs. Temporal progress of crystallization is represented as an increase in opacity of the W phase due to the presence of excipient.

FIG. 4. Stereomicroscopic images depicting; (a) controlled generation of O1/W/O2 double emulsion drops, (b), (c), (d) & (e) time lapse images of double emulsion droplet break-up, (f) collected double emulsions of the ‘n’-in-1 droplet morphology on a PDMS coated glass slide. All scale bars represent 300 μm.

FIG. 5 shows stereomicroscopic images depicting (a) collected double emulsions of the ‘n’-in-1 droplet morphology, (b)-(c) monodispersed ‘DE’ and ‘D²E’ SAs, respectively, which are the end products of crystallization.

FIG. 6. Schematic representation of the crystallization process and micrographs of ‘DE’ (Left Column) and ‘D2E’ (Right Column) formulations showing: initial shrinkage and generation of supersaturation prior to the formation of a sucrose shell at the onset of crystallization. Thereafter, either translucent SAs of the DE formulation or fully opaque SAs of the D2E formulation are obtained. All scale bars represent 100 μm.

FIG. 7. shows representative FESEM images, XRD characterization of spherical agglomerates from ‘DE’ and ‘D²E’ experiments. (a) SA of excipient (sucrose) and hydrophobic API (ROY) displaying a uniform and smooth surface, (b) SA of hydrophilic API (glycine), sucrose and ROY exhibiting a rough surface with crystals packed together with sucrose, (c) Close-up of faceted crystals located on the surface of the ‘D²E’ SAs, (d) XRD pattern of the ‘DE’ SAs showing peaks corresponding to the yellow prism and red plate polymorph of ROY, (e) XRD pattern of the ‘D²E’ SAs showing peaks corresponding to the yellow prism and red plate polymorph of ROY and γ-glycine.

FIG. 8. shows DSC profiles from (a) ‘D²E’ and (b) ‘DE’ experiments—characteristic peaks are found in the vicinity of 109° C. (ROY), 180° C.-192° C. (Sucrose) and 250° C. (Glycine) respectively.

FIG. 9. (a), (c) Optical and (b), (d) FESEM images of microparticles containing ROY and ethyl cellulose (EC) for the ‘thin’ (0.5 mm) and ‘thick’ (2 mm) film scenarios respectively. The drug-excipient loading ratio is 4:1 (320 mg of ROY: 80 mg of EC) for both cases. (e) Differential scanning calorimetry (DSC) profiles for the particles with exotherms at 106.9° C. and 112.7° C. corresponding to the yellow needle (YT04) and orange plate (OP) polymorphs of ROY respectively.

FIG. 10. Representative FESEM images of ROY-EC microparticles from thin and thick film experiments: (a), (b) YT04 particles displaying a compact, cellular structure with an EC scaffold harboring polycrystalline ROY domains, and (c), (d) Orange plate (OP) ROY particles displaying plate like crystals on the surface and within ‘the domains’ surrounded by a porous EC scaffold.

FIG. 11. (a)-(e) Time lapse stereomicroscopic images of emulsion drops subjected to evaporative crystallization under the thin film (0.5 mm) condition. Images were taken at intervals of 10 minutes each. (f) A phenomenological schematic capturing the various stages in the microparticle formation process, including solvent evaporation/shrinkage, liquid-liquid phase separation, EC scaffold formation and crystallization. All scale bars represent 100 μm.

FIG. 12. Optical [(a), (b)] and FESEM [(c), (d)] images of pure ROY (400 mg/mL) microparticles: a mixture of yellow, orange and brown colored particles indicate poor polymorphic selectivity; (e) Differential scanning calorimetry (DSC) profiles indicating concomitant polymorphism of YT04 and OP polymorphs of ROY for both thin (0.5 mm) and thick film (2 mm) conditions.

FIG. 13. Time-lapse stereomicroscopic images of ROY-DCM emulsion drops subjected to evaporative crystallization under the thin film (0.5 mm) condition. Droplet shrinkage followed by oiling out of ROY from DCM and subsequent crystallization was observed. All scale bars represent 100 μm.

FIG. 14. Optical and FESEM images of CBZ-EC microparticles from thin and thick film experiments: (a),(b),(d),(e) monodisperse population of particles with a smooth surface morphology and (c),(f) broken cross section of particle displaying needle shaped crystals embedded in the porous excipient matrix. (g) XRD patterns corresponding to form II and form III polymorphs of CBZ obtained for the thin and thick film cases respectively. (h) Differential scanning calorimetry (DSC) profiles for thin film and thick film cases: characteristic exotherms for form II and form III polymorphs of CBZ are found at 188° C. and 192° C. respectively.

### DETAILED DESCRIPTION

### Experimental Section

### Materials

Materials.

Glycine (>99%), dodecane (>99%), Span-80, trichloro-(1H,1H,2H,2H-perfluorooctyl)-silane (97%), (3-aminopropyl)triethoxysilane (97%), ammonium lauryl sulphate solution (ALS, 30% in water), n-hexane (HPLC grade, 95%) and mineral oil (light) were purchased from Sigma-Aldrich (Singapore) and used as received. Sodium dodecyl sulphate (SDS, 85%) was purchased from Merck (Germany). 5-methyl-2-[(2-nitrophenyl)amino]-3-thiophenecarbonitrile(ROY) was purchased from Nanjing Chemlin Chemical Industry Co. Ltd, China. Ethyl acetate (99.9%) was purchased from Fischer scientific (Singapore). Ultrapure water (18.3 MΩ) obtained using a Millipore Milli-Q purification system was used to prepare aqueous glycine solutions. Harvard PHD 22/2000 series syringe pump was used for regulated flow at μL scales. Square and cylindrical glass capillaries of ID 1 mm and 0.7 mm respectively were purchased from Arte glass associates Co., Ltd. Japan. Poly(vinyl) alcohol (PVA) (M.W.—67,000), dichloromethane (DCM) (99.5%), ethyl cellulose (EC) (viscosity 10 cP) and carbamazepine (CBZ) were purchased from Sigma-Aldrich (Singapore) and used as received.

### Methods

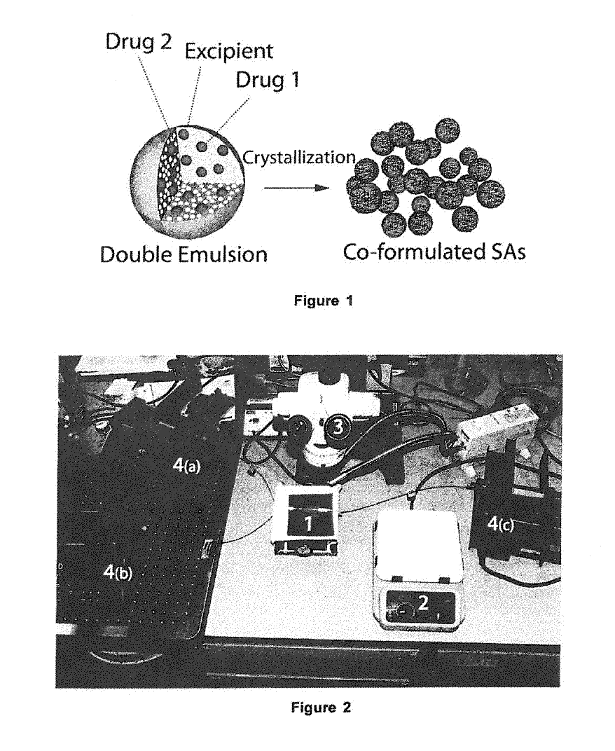

A photograph of the apparatus for working the invention is shown in FIG. 2. The setup consists of an emulsion generation apparatus, syringe pumps (Harvard PHD 22/2000 series), stereo microscope and heated surface has been assembled to demonstrate the capabilities of the invention.

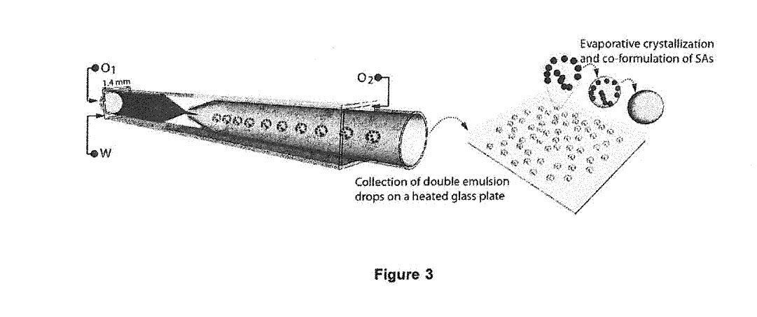

The emulsion generation apparatus is an assembly of three glass capillaries—two round and a square capillary—as presented by Weitz and co-workers (PCT Patent Appl. No. PCT/US2006/007772, incorporated herein by reference). A schematic of the apparatus depicting generation of O₁/W/O₂ double emulsions is shown in FIG. 3.

The axisymmetric coaxial glass capillary flow-focusing device was assembled using a square and two round capillaries. Round capillary 1 (C1) (colored red in FIG. 3) serves as the inlet for the inner fluid whilst round capillary 2 (C2) (colored yellow in FIG. 3) serves as the collection tube for the double emulsions. The round glass capillary collection nozzle (colored yellow in FIG. 3) and the square glass capillary are silanized to alter their wetting properties, specifically, for hydrophobicity and hydrophilicity, respectively. The square capillary was silanized with (3-aminopropyl)triethoxysilane (97%) and C2 was silanized with trichloro-(1H,1H,2H,2H-perfluorooctyl)-silane for hydrophilic and hydrophobic wetting properties respectively, to aid in double emulsion generation. 10 μL of either silane was used per glass capillary and silanization was carried out for a minimum of 8 hours in a vacuum chamber at a pressure of 0.08 MPa.

A total of 3 fluids (outer O₂, middle W and inner O₁) are infused into the emulsion generating device via the round glass dispensing nozzle (colored red in FIG. 3) and through the coaxial regions to form oil-in-water-in-oil (O₁/W/O₂) double emulsions. The O₁ and W phases carry the hydrophobic and hydrophilic APIs respectively while the O₂ phase serves as the continuous phase. The excipient, usually hydrophilic in nature, resides in the W phase.

The inner-most oil phase (O₁) was prepared by mixing 1 parts ROY (30 mg/mL) in ethyl acetate solution with 5 parts dodecane containing 0.3% (w/w) surfactant, Span 80. Middle aqueous phase (W) was prepared by dissolving 1 g of sucrose, 100 mg of glycine and 100 mg of surfactant (SDS) in 5 mL ultra-pure water for the D2E formulation. Light mineral oil with 0.5% (w/w) of surfactant (Span80), was used as the continuous phase (O₂).

In the specific embodiment of the invention described herein, O₂ and W phases were infused from the two ends of the square capillary through the outer coaxial region while O₁ phase was infused through C1 using syringe pumps (Harvard PHD 22/2000 series). However, the skilled man will appreciate that the various phases can be infused through the apparatus in different manners.

The flow rates of these phases can be tuned to adjust the size of each of the liquid domains (i.e. O₁ and W phase) and thus achieve the desired loading of each API. The typical operating flow rates follow a decreasing trend in the order of O₂, W and O₁ respectively. Specifically, the flow rates of 40 μL/min, 7 μL/min and 1.8 μL/min were used for the O₂, W and O₁ phases respectively.

All the three fluids were hydrodynamically flow focused through the nozzle of C2 resulting in the formation of the double emulsion drops. Approximately 1 mL of the double emulsion was collected on a glass slide spun coated with a thin layer of polydimethylsiloxane (PDMS) and subsequently heated to a temperature of 80-100° C., typically 90° C. on a hot plate (Thermo Scientific CIMAREC) for evaporative crystallization resulting in the formation of the formulated spherical agglomerates (SAs) of ˜200 μm. High-speed real-time imaging of the droplet breakup and stable emulsions collected on the glass slide was performed with high speed digital cameras (Basler pI640 or Miro Phantom EX2) mounted onto a stereomicroscope (Leica MZ16). A Leica CLS 150 XE light source was used.

For the purpose of exemplification, we prepared two types of formulations with the flow setup. We formulated a hydrophobic API (Drug) in a hydrophilic excipient (E) matrix (DE formulation) in our first exemplification and formulated a hydrophobic API (Drug) in a hydrophilic matrix (E) containing excipient and a hydrophilic API (D²E formulation) in the D²E exemplification.

In each exemplification, we carried out high-speed imaging with high speed digital cameras mounted on a stereomicroscope to document the operation of the emulsion generating device. The SAs of each formulation were characterized by using microscopic image analysis, field emission scanning electron microscopy (FE-SEM), powder X-ray diffraction (PXRD) and differential scanning calorimetry (DSC). For the size distribution studies we used an inverted microscope (Nikon Eclipse Ti) operated in bright field mode. The inbuilt software (NIS Elements 3.22.0) was used to measure the diameters of the agglomerates (circle by three points method) and to estimate the average diameters and standard deviations based on measurements of at least 100 SAs.

A field emission scanning electron microscope (JEOL JSM-6700F) at 5 kV accelerating voltage was used to acquire further structural information on the SAs. All samples were prepared on conventional SEM stubs with carbon tape and were coated with ˜10 nm of platinum by sputter coating. An XRD diffractometer (LabX XRD-6000, Shimadzu) with characteristic Cu radiation was used for polymorphic characterization. The X-ray diffractometer was operated at 40 kV, 30 mA and at a scanning rate of 2°/min over the range of 10-40°, using the Cu radiation wavelength of 1.54 Å. The DSC thermograms were obtained using a Mettler Toledo DSC 882 apparatus. Around 5 or 10 mg of sample was crimped in a sealed aluminium pan and heated at 5° C./min in the range of room temperature to 225° C. or 280° C. using an empty sealed pan as reference. Dry nitrogen was used as purge gas and the N2 flow rate was 50 μL/min. GC analysis was carried out on a Shimadzu GC 2010 Plus apparatus equipped with an auto injector (AOC-20i), flame-ionization detector and a separation column (30 m, i.d. 0.25 mm). Around 10 mg of sample was crushed and added to 1 mL of hexane and loaded into the GC. The system was run for 8 minutes from 50 to 250° C. for a helium gas purge flow of 3 mL/min.

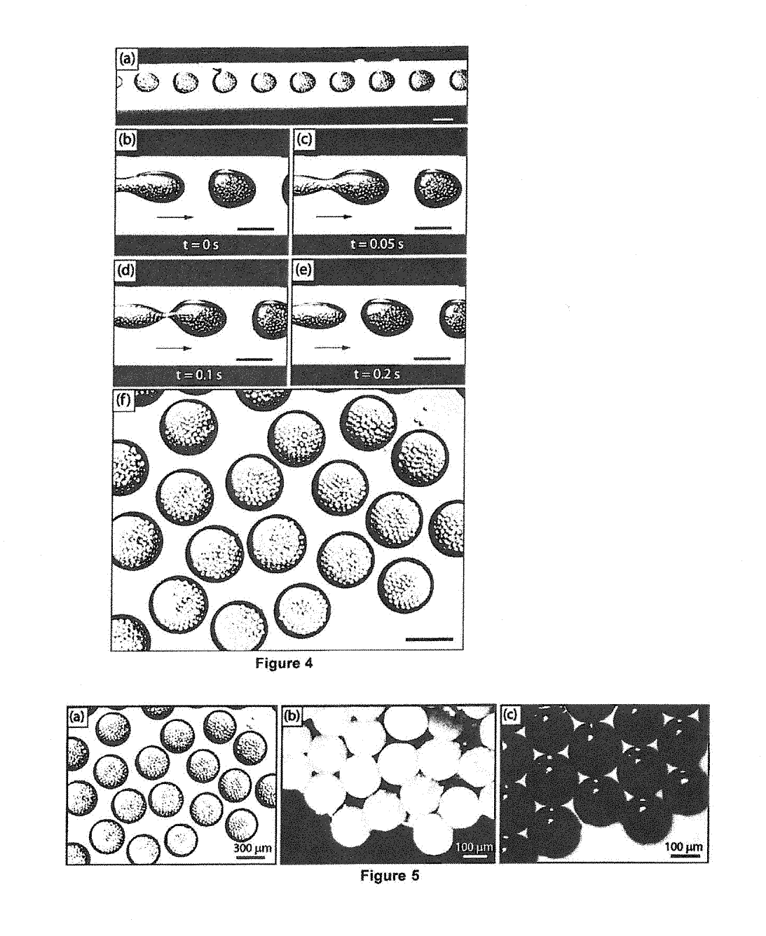

We observed droplet generation in the microfluidic device using high speed imaging. A uniform stream of double emulsions with multiple inner droplets (n-in-1) (FIG. 4a) is observed while operating in the jetting regime where droplet formation occurs downstream of the circular orifice of the collection tube. Jetting is a result of dominant viscous effects over inertial effects and interfacial forces; the viscosity of the outer O² phase is ˜30 times that of the middle W phase. The system operates at a low Reynolds number, as is characteristic of most microfluidic flow scenarios. The transition from the dripping to jetting regime is described by a Capillary number

$$Ca = \frac{μ V}{γ} ∼ 1 ,$$

where μ is the viscosity of the O² phase, V is a mean velocity of the inner W phase and γ is the interfacial tension between the O² and W phases. The size of the middle and the inner phase droplets can be tuned by varying the flow rates of the respective fluids. The volumetric flow rates of the O2, W and O1 phases were set to 40, 7 and 1.8 μL/min respectively. At these flow conditions, the frequency of droplet generation is 5 droplets per second (FIGS. 4b to 4e).

Analysis using high speed imaging reveals double emulsions of a mean diameter of 382 μm (FIGS. 4f & 5a) with a standard deviation of 2%. A count of the number of inner O₁ droplets within these double emulsions gives ‘n’=85±8 droplets. The diameter of the inner O₁ droplets is ˜25 μm. By calculating the total volume of the O1 droplets and the volume of the W phase, we estimate that ˜45% of the droplet volume is occupied by the O1 phase. A typical SA of the drug-drug-excipient (‘D²E’) formulation contains 1.3 μg of sucrose, 0.13 μg of glycine and 0.03 μg of ROY, yielding a loading ratio of 40:4:1 (Sucrose/Glycine/ROY). Similarly, SAs of the drug-excipient (‘DE’) formulation yield a loading ratio of 40:1 (Sucrose/ROY).

The presence of the O¹ and W phases allows for hydrophobic and hydrophilic APIs to be formulated as a single entity; a challenging task in contemporary pharmaceutical processing. The loading ratio of the APIs can also be monitored and controlled accurately. The concentration of the API in the O¹ or W phase can be regulated to increase or decrease the drug loading while the droplet morphology remains fixed. Alternatively, the loading can also be adjusted by altering the number of O¹ droplets or by varying the overall diameter of the double emulsion droplet.

We were able to fabricate monodispersed SAs of both drug-excipient (‘DE’) and drug-drug-excipient (‘D²E’) types with tunable particle sizes in the 100-300 μm diameter range. Under the specific flow conditions mentioned earlier in the description, the mean particle size of the ‘DE’ (FIG. 5b) and ‘D²E’ (FIG. 5c) SAs were ˜200 μm diameter with a standard deviation of <5%. This approach to monodisperse particulate formulations potentially circumvents several drawbacks in conventional processing, such as wide size distribution in batch crystallization, de-mixing in blending and challenges in the co-formulation of hydrophobic and hydrophilic APIs and excipients.

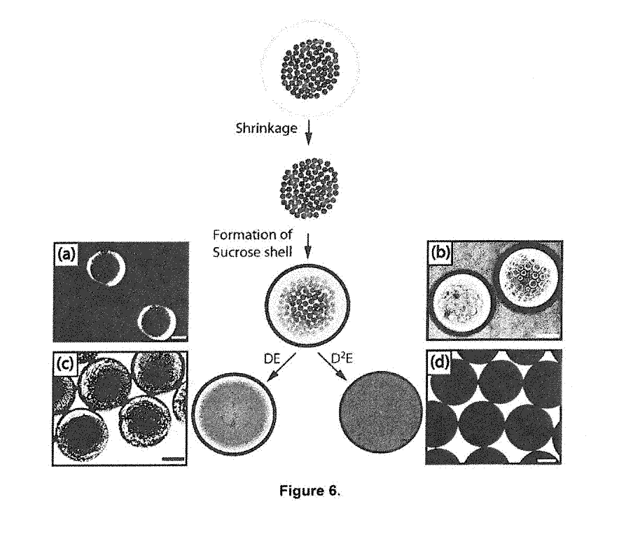

We observed several stages in the process of crystallization. Firstly, the double emulsion droplets shrank to ˜60% of their original droplet diameter. Thereafter, a hard and brittle shell was observed to form at the W/O² interface, encapsulating the inner droplets (FIGS. 6a and 6b). Stereomicroscopic images obtained during the course of crystallization show the formation of a sucrose shell while the O¹ droplets are still present. Encapsulation is crucial in ensuring entrapment of the hydrophobic API in the event of coalescence of O¹ droplets with the O² phase. An increase in opacity of the encapsulated emulsions followed. The ‘D²E’ SAs appeared opaque due to the presence of glycine while those of the ‘DE’ SAs appeared translucent (FIGS. 6c and 6d).

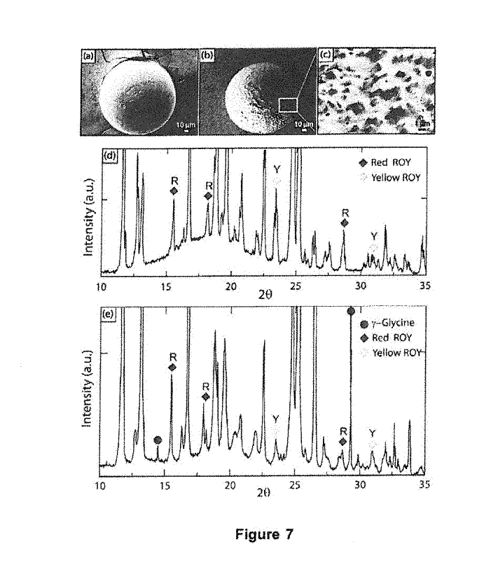

Electron microscopy revealed that the surface of the ‘DE’ SAs was smooth while that of the ‘D²E’ SAs was coarse (FIGS. 7a and 7b). The smooth texture of the ‘DE’ SAs is expected as it is typical of formulations containing sucrose. On closer observation (FIG. 7c), crystal facets of ˜2 μm were observed to populate the surface of the ‘D²E’ SAs; these facets can be attributed to the presence of hydrophilic API in the excipient matrix.

XRD reveals the presence of γ-glycine and the red and yellow polymorphs of ROY respectively, as indicated in FIGS. 7d and 7e; the observed characteristic peak for ROY at 15.6°, 18.2° and 23.8°, which are the major peaks in bulk ROY, provides strong validation for its presence within the SAs. XRD characterization revealed that the yellow prism (Y) polymorph was the major component. Interestingly, we obtained γ-glycine in our D2E formulations, as opposed to the more commonly obtained α-glycine in emulsion-based crystallization.15 This can be attributed to the role of the sodium ions present in the surfactant used—sodium dodecyl sulfate (SDS). Sodium ions have been reported to inhibit the growth of metastable α-glycine via interaction with the carboxylate group of glycine zwitterions in solution, thus promoting the growth of γ-polymorph.29 Control experiments using a different surfactant—ammonium lauryl sulfate (ALS) yielded α-glycine (refer Supporting Information, Section 2), thus confirming the role of SDS in the formation of γ-glycine and thus demonstrating the possibility of polymorphic control using surfactants as additives.

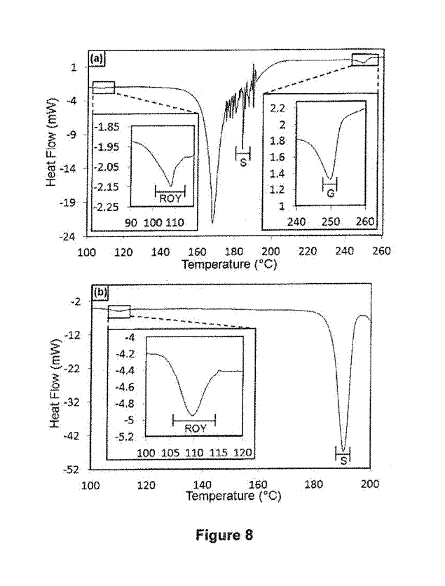

From the DSC thermograms (FIGS. 8a and 8b), an exotherm at 109° C. affirms the presence of ROY and the exotherm at 250° C. confirms the presence of glycine. The characteristic region of peaks observed between 180° C. to 192° C., correspond to the range that defines the decomposition temperature of sucrose. The exotherm observed at 160-170° C. for the ‘D2E’ trials may be attributed to the decomposition temperature of glucose—a product of the hydrolysis of sucrose and precursor to the Maillard reaction. The peak position is characteristic of glucose decomposition for heating rates of 2-10° C./min.30 Lastly, the exotherm at 250° C. confirms the presence of glycine. The DSC results reinforce the XRD results thus affirming successful co-formulation of the two API models.

In addition, we also studied the levels of residual solvent in the formulated SAs using GC analysis. Dodecane is the major component of the inner organic phase O¹, and its residual amount in the SAs was measured to be 7.5 μg/mg of SAs. This is well within the acceptable limits of residual solvents on typical paraffins under class 3 classification of residual solvents.

### Further Evaporation Studies

The aqueous continuous phase (W) was prepared by mixing 1.5% wt PVA in water. The dispersed phase (O) was one of the following three: (i) ROY in DCM (400 mg/mL), (ii) ROY-EC in DCM (320 and 80 mg/mL, respectively), (iii) CBZ-EC in DCM (240 and 60 mg/mL, respectively). W and O phases were infused from the two ends of the square capillary through the outer coaxial region using syringe pumps (Harvard PHD 22/2000 series) at flow rates of 150 and 50 μL/min respectively. The fluids were hydrodynamically flow focused through the nozzle of the round capillary resulting in the formation of the emulsion drops. 3.7 cm ID glass wells were used for sample collection and as crystallization platforms. Approximately 100 μL of O/W emulsions were dispensed directly into the glass well containing either a ‘thin’ (0.5 mm) or ‘thick’ (2 mm) film of the continuous phase. Evaporative crystallization was performed at room temperature (24° C.) and at ambient humidity (55%). Optical microscopy images were captured using a Qimaging MicroPublisher 5.0 RTV camera mounted on an Olympus SZX7 microscope. A Leica CLS 150 XE light source was used. A thin film of continuous phase persisted at the end of all experiments.

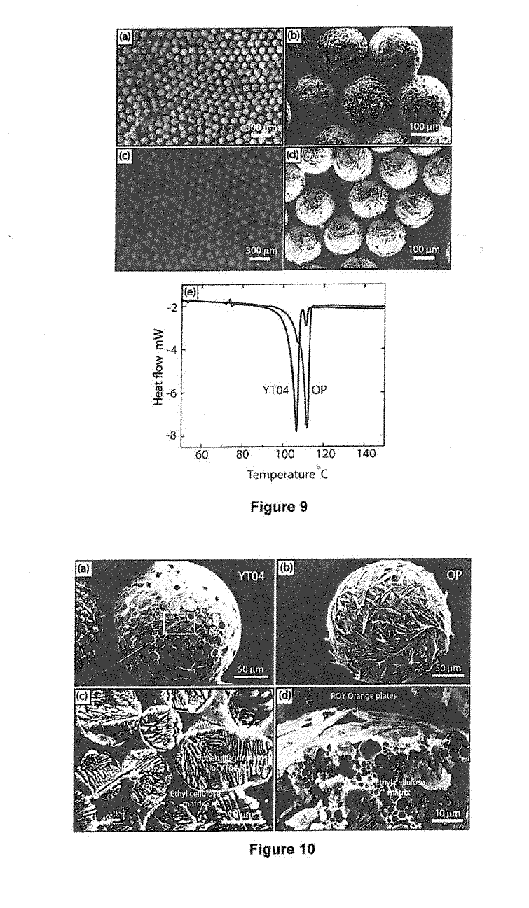

Emulsions of ROY-EC in DCM (100 μL) were dispensed into a glass well containing a pre-dispensed film of water-PVA solution (0.5 and 2 mm nominal film thickness) for subsequent evaporative crystallization. The entire crystallization process took ˜40 min and ˜4 hours for thin and thick film cases respectively, at ambient temperature (24° C.). Monodisperse SAs of ROY-EC of diameter 180 μm (with a standard deviation of 5%) were produced under both conditions. Polymorphic selection of nearly 100% was achieved for both conditions, as indicated by particle color and the DSC characterization (FIG. 9); yellow and orange microparticles were obtained for the thin and thick film cases respectively. DSC characterization reveals the yellow polymorph to be YT04 and the orange polymorph to be orange plate (OP); here we note that YT04 is thermodynamically less stable than OP among the reported polymorphs of ROY at room temperature. FIG. 10 compares electron microscopy (FESEM) images of the structure of YT04 and OP SAs, highlighting the spherical shape of particles obtained in both cases. Further, FESEM images also reveal interesting structural differences between the two cases. YT04 particles have a compact structure that consists of polycrystalline, presumably spherulitic domains tightly embedded within an EC matrix, whereas OP particles exhibit a void-filled porous structure with large single crystals loosely encapsulated within the pores and ribbon-like crystal flakes covering the particle surfaces.

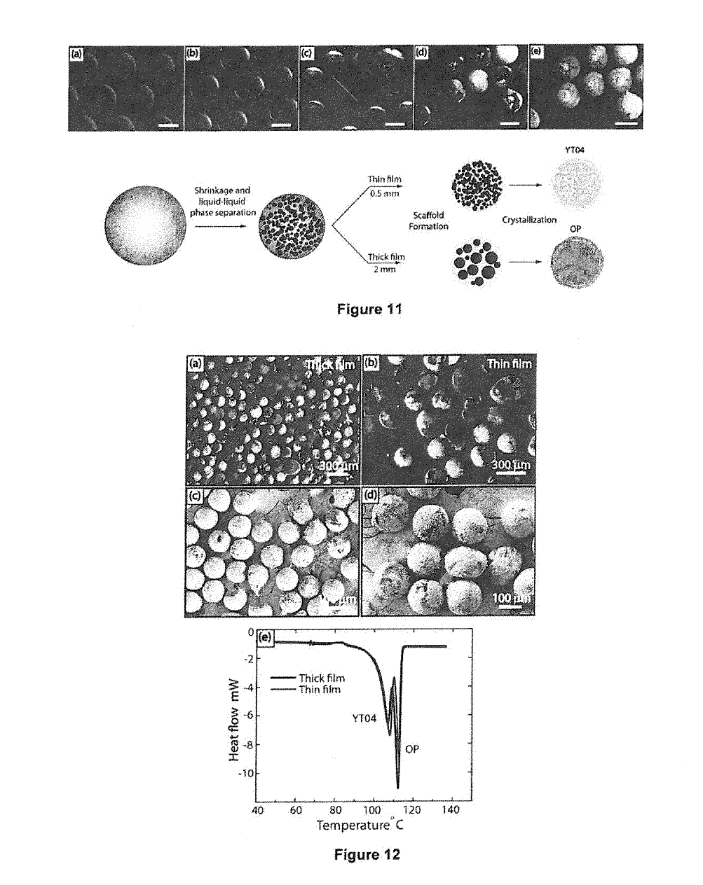

To better understand the particle formation process, we conducted online optical microscopic monitoring of the entire crystallization process. As shown in FIG. 11(a)-(e), which are time-lapse optical microscopic images of evaporating emulsion drops, we noted the occurrence of a liquid-liquid phase separation of the three component (ROY-EC-DCM) system as the solvent (DCM) evaporates. Small droplets (‘domains’) were observed to form within the dispensed droplets and grow in size over time. The average domain size measured immediately after the first crystallization event in the droplet ensemble for the thin film case (3 μm) was smaller than that observed in the thick film experiments (12 μm), indicating coarsening of the domains in the latter case. As suggested by the FESEM images in FIG. 10, ROY crystals formed the major component in these domains whereas EC formed an interconnected scaffold surrounding the domains.

We interpret and explain our observations in terms of an interplay between simultaneous dynamic processes occurring within the evaporating emulsion drops containing API-excipient mixtures—(i) liquid-liquid phase separation of the three component system, API-excipient-solvent, due to solvent evaporation, which compartmentalizes the API rich solution into small domains surrounded by the excipient, which then provide surfaces for heterogeneous nucleation of the API and (ii) increasing supersaturation of both the API and excipient rich phases, eventually leading to solidification of the excipient, which further facilitates crystallization of API. In the thin film case, due to fast evaporation and supersaturation generation, the domains form rapidly (within ˜3 minutes), resulting in a population of highly supersaturated internal droplets, where conditions are conducive to spherulitic growth. On the other hand, in the case of thick films, slow evaporation results in a milder temporal supersaturation profile and the possible coarsening of the domains. Our observation is of the less stable YT04 polymorph25 appearing at higher evaporation rates and the comparatively more stable OP polymorph crystallizing under a slow rate of supersaturation generation in confined spaces.

### Role of the Excipient in Evaporation

To further investigate and validate the role of the excipient, we compared and contrasted the above results with the case of ROY crystallization in the absence of EC. 100 L of emulsions generated from ROY-DCM solution in aqueous PVA solution were dispensed into a glass well containing a pre-dispensed film of water-PVA solution (0.5 and 2 mm film thickness) for subsequent evaporative crystallization. As compared to the case with excipient, relatively fewer monodisperse and irregular shaped particles of ROY were formed under both the thin and thick film conditions (FIG. 12). The time taken for particle formation was ˜1.5 hours and ˜7 hours for the thin and thick film cases, respectively, which is 2-3 times longer than the above cases where EC was used along with ROY. Optical microscopy images of the ROY microparticles indicate concomitant polymorphism and thus poor control over polymorphic selection (FIG. 12); DSC characterization further confirms the concomitant occurrence of both YT04 and OP polymorphs.

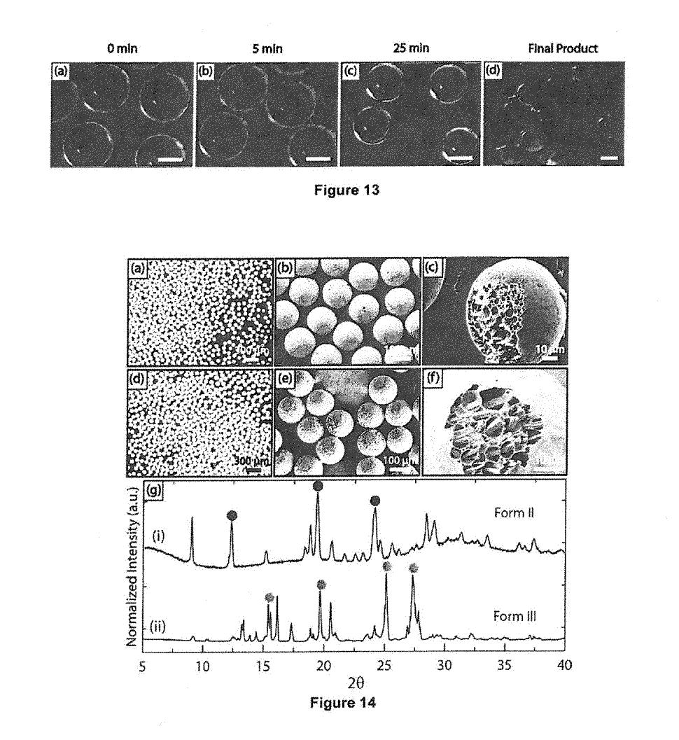

Liquid-liquid phase separation was also observed in this case (FIG. 13); small ROY precipitates were seen appearing and growing inside the emulsion droplets within ˜1 min after dispensing. In the case of an API-solvent system, this phase separation is known as ‘oiling out’, and is commonly observed during the crystallization of small organic molecules. Here, the solute-solvent system transitions from a single liquid phase into a metastable liquid-liquid state (having a solute-rich and solute-lean phase), bypassing the solid-liquid zone in the phase diagram altogether. Recent pharmaceutical development has seen an increase in the number of lipophilic and non-polar API molecules, such as ROY, which do not easily self-assemble, and are prone to liquid-liquid phase separation. Further, the metastable liquid-liquid state is known to hinder primary and secondary nucleation, leading to long crystallization process times of up to 35 hours; often, special measures are needed to move the system away from this part of the phase diagram to promote nucleation and growth of crystals. This is in keeping with our observations of longer crystallization times for this case, as compared to the results with excipient. In the latter case, the formation of an excipient scaffold upon solvent evaporation provided heterogeneous sites for nucleation of ROY crystals in both the thin and thick film cases, the polymorphism ultimately being dictated by the different temporal rates of supersaturation generation.

Finally, to validate the core idea, we demonstrate controlled polymorphic selection of another model molecule—carbamazepine (CBZ)—an anticonvulsant which has multiple polymorphic forms via conformational polymorphism. An analogous protocol was followed in this case; droplets of CBZ in DCM were generated in an aqueous PVA continuous phase, and subjected to evaporative crystallization in both thin (0.5 mm) and thick films (2 mm), as for the case of ROY. The particles generated were highly monodisperse and had a smooth surface morphology. Electron microscopy of broken sections of the particles show needle shaped CBZ crystals trapped within the porous framework of ethyl cellulose (FIG. 14) in both cases. Powder X-Ray diffraction (XRD) characterization reveals that particles from the thin and thick film experiments correspond to least stable form II and most stable form III polymorphs of CBZ (Form II<IV<I<III) respectively. As indicated in FIG. 14g [(i) and (ii)], major peaks identified at 13.26°, 18.56°, and 24.54° are attributed to form II CBZ and peaks at 15.36°, 19.56°, 25.00°, and 27.47° to form III CBZ respectively. Dominant and unrepeated occurrence of characteristic peaks corresponding to the two forms of CBZ provides strong evidence of polymorphic selection using our method. Finally, DSC analysis was performed to validate polymorphic selection of carbamazepine (CBZ) determined by XRD, showing Form II under thin film and Form III under thick film. DSC thermograms were recorded at 5° C./min from 25 to 225° C., corresponding to the melting range of CBZ forms (FIG. 14h). An exotherm at 188° C. for the sample from the thin film experiment confirmed the presence of form II CBZ polymorph, which has a reported melting point in the range of 188-192° C. The exotherm at 192° C. for CBZ samples from the thick film experiment confirmed the presence of form III CBZ polymorph, which has a reported melting point in the range of 189-193° C.18 DSC thermograms thus reinforced our XRD results, providing strong validation for polymorphic selection of CBZ.

### CONCLUSION

The invention described herein overcomes the challenges faced in pharmaceutical formulations wherein we demonstrate a single step formulation platform for the fabrication of monodispersed microparticles of ˜200 μm size containing crystals of a hydrophobic model API (ROY) embedded within a hydrophilic excipient (sucrose) matrix (‘DE’ formulation), which in turn may also contain a hydrophilic model API (glycine) (‘D²E’ formulation).

We have shown a pharmaceutical formulation process in ‘bottom-up’ fashion, where crystallization and formulation occur in tandem, instead of via energy intensive ‘top-down’ processes in traditional manufacturing. To do this we have leveraged emulsion-based spherical crystallization and microfluidic capillary-based emulsification. We dispense the components of the formulation into monodispersed oil-in-water-in-oil (O₁/W/O₂) or water-in-oil-in-water (W₁/O/W₂) double emulsions using capillary microfluidics and spherically crystallize them to form exemplary DE and D²E microparticles—the first demonstration of its kind. The method also has capabilities to completely circumvent several energy-intensive and ubiquitously batch processes in traditional manufacturing, thereby offering the potential for continuous, sustainable pharmaceutical crystallization coupled with advanced formulations.

1. A method for the manufacture of a pharmaceutical product comprising more than one pharmacologically active ingredient, comprising:
i) dispersing a first pharmacologically active ingredient in a first fluid;
ii) dispersing a second pharmacologically active ingredient and an excipient or carrier in a second fluid;
iii) mixing said first and second fluids with a carrier fluid to form a multiple emulsion; and
iv) crystallizing the emulsion by heating to form spherical agglomerates.

2. The method according to claim 1 wherein said first pharmacologically active ingredient and second pharmacologically active ingredient are either hydrophobic or hydrophilic.

3. The method according to claim 2 wherein said first pharmacologically active ingredient is hydrophobic and said second pharmacologically active ingredient is hydrophilic.

4. The method according to claim 2 wherein said first pharmacologically active ingredient is hydrophilic and said second pharmacologically active ingredient is hydrophobic.

5. The method according to claim 2 wherein said hydrophobic pharmacologically active ingredient is dispersed in a compatible non-aqueous first fluid.

6. The method according to claim 2 wherein said hydrophilic pharmacologically active ingredient is dispersed in a compatible aqueous second fluid.

7. The method according to claim 2 wherein said excipient or carrier is dispersed in a compatible non-aqueous or aqueous fluid.

8. The method according to claim 1 wherein at least one further fluid is provided containing a dispersion of at least one further pharmacologically active ingredient.

9. The method according to claim 1 wherein said mixing includes passing the said first fluid, second fluid, and carrier fluid through a microfluidic device.

10. The method according to claim 9 wherein each said first fluid, second fluid, and carrier fluid is introduced into the microfluidic device via a different channel each one of which converges at a mixing point where the said channels are brought together.

11. The method according to claim 10 wherein the rate of flow of said first fluid, second fluid, and carrier fluid through said microfluidic device is controlled or regulated in accordance with the desired formulation of the first pharmacologically active ingredient and the second pharmacologically active ingredient.

12. The method according to claim 1 wherein part iv) includes collecting the emulsion on a heated surface at a selected film thickness and allowing the emulsion to crystalize to form spherical agglomerates.

13. The method according to claim 12 wherein said selected film thickness is between 0.5-2 mm.

14. The method according to claim 1 wherein said steps of dispersing, mixing, and crystallizing are performed under conditions providing spherical agglomerates having a mean diameter in a range of about 100-300 μm.

15. The method according to claim 14 wherein said steps of dispersing, mixing, and crystallizing are performed under conditions providing spherical agglomerates having a mean diameter of about 200 μm.

16. A crystalized spherical agglomerate (SA) manufactured according to the method of claim 1 comprising at least two pharmacologically active ingredients (APIs) and an excipient or carrier.

17. A crystalized spherical agglomerate (SA) comprising at least two pharmacologically active ingredients (APIs) and an excipient or carrier.

18. The crystalized spherical agglomerate (SA) according to claim 17 wherein said SA comprises at least one further pharmacologically active ingredient dispersed in an excipient or carrier.

19. The crystalized spherical agglomerate (SA) according to claim 17 wherein said spherical agglomerate (SA) comprises particles having a mean diameter of about 100-300 μm.

20. The crystalized spherical agglomerate (SA) according to claim 19 wherein said spherical agglomerate (SA) comprises particles having a mean diameter of about 200 μm.

21. A pharmaceutical product comprising the crystalized spherical agglomerate (SA) according to claim 17.
